(19) United States
(12) Patent Application Publication
(10) Pub. No.: US 2015/0024962 A1
(43) Pub. Date: Jan. 22, 2015

(54) MARKER SEQUENCES FOR MULTIPLE SCLEROSIS AND USE THEREOF

(71) Applicant: PROTAGEN AG, Dortmund (DE)

(72) Inventors: Jens Beator, Kreiensen (DE); Angelika Lüking, Bochum (DE); Axel Kowald, Bochum (DE); Helmut Meyer, Recklinghausen (DE)

(21) Appl. No.: 14/453,340

(22) Filed: Aug. 6, 2014

Related U.S. Application Data
(63) Continuation of application No. 12/676,178, filed on Jun. 21, 2010, now abandoned.

(30) Foreign Application Priority Data
Sep. 3, 2007 (DE) 102007041657.3

(51) Int. Cl. G01N 33/564

(52) U.S. Cl. CPC G01N 33/564; G01N 2800/285
USPC 506/9

(57) ABSTRACT

The present invention relates to new marker sequences for multiple sclerosis and the diagnostic use thereof together with a method for screening of potential active substances for multiple sclerosis by means of these marker sequences. Furthermore, the invention relates to a diagnostic device containing such marker sequences for multiple sclerosis, in particular a protein biochip and the use thereof.

### RELATED APPLICATIONS

This application is a continuation of application Ser. No. 12/676,178, filed Jun. 21, 2010, and the entire contents of which is incorporated by reference herein. Application Ser. No. 12/676,178 is a national stage application (under 35 U.S.C. §371) of PCT/DE2008/001546, filed Sep. 3, 2008, and claims the benefit of German application 102007041657.3, filed Sep. 3, 2007.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_Listing_{—}14462_{—}00014_US.txt. The size of the text file is 1, 643 KB, and the text file was created on Aug. 6, 2014.

The present invention relates to new marker sequences for multiple sclerosis and the diagnostic use thereof together with a method for screening potential active substances for multiple sclerosis by means of these marker sequences. Furthermore, the invention relates to a diagnostic device containing marker sequences of this type for multiple sclerosis, in particular a protein biochip and the use thereof.

Protein biochips are gaining increasing industrial importance in analysis and diagnosis as well as in pharmaceutical development. Protein biochips have become established as screening instruments.

The rapid and highly parallel detection of a multiplicity of specifically binding analysis molecules in a single experiment is rendered possible hereby. To produce protein biochips, it is necessary to have the required proteins available. For this purpose, in particular protein expression libraries have become established. The high throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P., (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome Version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, an approach of this type is strongly connected to the progress of the genome sequencing projects and the annotation of these gene sequences. Furthermore, the determination of the expressed sequence can be ambiguous due to differential splicing processes. This problem may be circumvented by the application of cDNA expression libraries (Büissow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C, Lehrach, H. and Walter, G. (2000) A human cDNA library for high-throughput protein expression screening. Genomics, 65, 1-8; Holz, C, Lueking, A., Bovekamp, L., Gutjahr, C, Bolotina, N., Lehrach, H. and Cahill, D. J. (2001) A human cDNA expression library in yeast enriched for open reading frames. Genome Res, 11, 1730-1735; Lueking, A., Holz, C, Gotthold, C, Lehrach, H. and Cahill, D. (2000) A system for dual protein expression in Pichia pastoris and Escherichia coli, Protein Expr. Purif, 20, 372-378). The cDNA of a particular tissue is hereby cloned into a bacterial or an eukaryotic expression vector, such as, e.g., yeast. The vectors used for the expression are generally characterized in that they carry inducible promoters that may be used to control the time of protein expression. Furthermore, expression vectors have sequences for so-called affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

For example, the gene products of a cDNA expression library from human fetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from the library could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Büssow (2000) supra; Lueking, A., Horn, M., Eickhoff, H., Büssow, K., Lehrach, H. and Walter, G. (1999) Protein microarrays for gene expression and antibody screening. Analytical Biochemistry, 270, 103-111). Protein biochips of this type based on cDNA expression libraries are in particular the subject matter of WO 99/57311 and WO 99/57312.

Furthermore, in addition to antigen-presenting protein biochips, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

However, there is a great need to provide indication-specific diagnostic devices, such as a protein biochip.

Marker sequences and the diagnostic use thereof for multiple sclerosis, in particular in the embodiment of a protein biochip, as well as tests in this regard for the screening of active substances have not been described in the prior art.

The object of the present invention is therefore to provide marker sequences and their diagnostic use.

The provision of specific marker sequences permits a reliable diagnosis and stratification of patients with multiple sclerosis, in particular by means of a protein biochip.

The invention therefore relates to the use of marker sequences for the diagnosis of multiple sclerosis, wherein at least one marker sequence of a cDNA selected from the group SEQ 1-395 or respectively a protein coding therefor or respectively a partial sequence or fragment thereof (hereinafter: marker sequences according to the invention) is determined on or from a patient to be examined.

It was possible to identify the marker sequences according to the invention by means of differential screening of samples from healthy test subjects with patient samples with multiple sclerosis.

The term “multiple sclerosis (MS), also encephalomyelitis disseminata)” is defined, e.g., according to Pschyrembel, de Gruyter, 261st edition (2007), Berlin and relates to an autoimmune inflammatory/demyelinating and degenerative disorder of the central nervous system.

In a further embodiment at least 2 to 5 or 10, preferably 30 to 50 marker sequences or 50 to 100 or more marker sequences are determined on or from a patient to be examined.

In a particular embodiment of the invention, the marker sequences of the SEQ 1-20 are particularly preferred, the marker sequences SEQ 21-50 are preferred, and furthermore the marker sequences SEQ 51-100 are preferred.

In a further embodiment of the invention, the marker sequences SEQ 1-10 and SEQ 11-20, as well as preferably SEQ 21-30, SEQ 31-40 or SEQ 41-50 are respectively particularly preferred.

In a further embodiment of the invention, the marker sequences according to the invention can likewise be combined, supplemented, fused or expanded likewise with known biomarkers for this indication.

In a preferred embodiment, the determination of the marker sequences is carried out outside the human body and the determination is carried out in an ex vivo/in vitro diagnosis.

In a further embodiment of the invention, the invention relates to the use of marker sequences as diagnostic agents, wherein at least one marker sequence of a cDNA is selected from the group SEQ 1-395 or respectively a protein coding therefor or respectively a partial sequence or fragment thereof.

Furthermore, the invention relates to a method for the diagnosis of multiple sclerosis, wherein a.) at least one marker sequence of a cDNA selected from the group SEQ 1-395 or respectively a protein coding therefor or respectively a partial sequence or fragment thereof is applied to a solid support and b.) is brought into contact with body fluid or tissue extract of a patient and c.) the detection of an interaction of the body fluid or tissue extract with the marker sequences from a.) is carried out.

The invention therefore likewise relates to diagnostic agents for the diagnosis of multiple sclerosis respectively selected from the group SEQ 1-395 or respectively a protein coding therefor or respectively a partial sequence or fragment thereof.

The detection of an interaction of this type can be carried out, for example, by a probe, in particular by an antibody.

The invention therefore likewise relates to the object of providing a diagnostic device or an assay, in particular a protein biochip, which permits a diagnosis or examination for multiple sclerosis.

Furthermore, the invention relates to a method for the stratification, in particular risk stratification and/or therapy control of a patient with multiple sclerosis, wherein at least one marker sequence of a cDNA selected from the group SEQ 1-395 or respectively a protein coding therefor is determined on a patient to be examined.

Furthermore, the stratification of the patients with multiple sclerosis in new or established subgroups of multiple sclerosis is also covered, as well as the expedient selection of patient groups for the clinical development of new therapeutic agents. The term therapy control likewise covers the allocation of patients to responders and non-responders regarding a therapy or the therapy course thereof.

“Diagnosis” for the purposes of this invention means the positive determination of multiple sclerosis by means of the marker sequences according to the invention as well as the assignment of the patients to multiple sclerosis. The term diagnosis covers medical diagnostics and examinations in this regard, in particular in-vitro diagnostics and laboratory diagnostics, likewise proteomics and nucleic acid blotting. Further tests can be necessary to be sure and to exclude other diseases. The term diagnosis therefore likewise covers the differential diagnosis of multiple sclerosis by means of the marker sequences according to the invention and the prognosis of multiple sclerosis.

“Stratification or therapy control” for the purposes of this invention means that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof.

In a further embodiment of the invention, the term “stratification” covers in particular the risk stratification with the prognosis of an outcome of a negative health event.

Within the scope of this invention, “patient” means any test subject—human or mammal—with the proviso that the test subject is tested for multiple sclerosis.

The term “marker sequences” for the purposes of this invention means that the cDNA or the polypeptide or protein that can be respectively obtained therefrom are significant for multiple sclerosis. For example, the cDNA or the polypeptide or protein that can be respectively obtained therefrom can exhibit an interaction with substances from the body fluid or tissue extract of a patient with multiple sclerosis (e.g., antigen (epitope)/antibody (paratope) interaction). For the purposes of the invention “wherein at least one marker sequence of a cDNA selected from the group SEQ 1-395 or respectively a protein coding therefor or respectively a partial sequence or fragment thereof is determined on a patient to be examined” means that an interaction between the body fluid or tissue extract of a patient and the marker sequences according to the invention is detected. An interaction of this type is, e.g., a bond, in particular a binding substance on at least one marker sequence according to the invention or in the case of a cDNA the hybridization with a suitable substance under selected conditions, in particular stringent conditions (e.g., such as usually defined in J. Sambrook, E. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology,” Green Publishing Associates and Wiley Interscience, N. Y. (1989)). One example of stringent hybridization conditions is: hybridization in 4×SSC at 65° C. (alternatively in 50% formamide and 4×SSC at 42° C.), followed by several washing steps in 0.1×SSC at 65° C. for a total of approximately one hour. An example of less stringent hybridization conditions is hybridization in 4×SSC at 37° C., followed by several washing steps in 1×SSC at room temperature.

According to the invention, substances of this type are constituents of a body fluid, in particular blood, whole blood, blood plasma, blood serum, patient serum, urine, cerebrospinal fluid, synovial fluid or of a tissue extract of the patient.

In a further embodiment of the invention, however, the marker sequences according to the invention can be present in a significantly higher or lower expression rate or concentration that indicates multiple sclerosis. The relative sick/healthy expression rates of the marker sequences for multiple sclerosis according to the invention are hereby determined by means of proteomics or nucleic acid blotting.

In a further embodiment of the invention, the marker sequences have a recognition signal that is addressed to the substance to be bound (e.g., antibody, nucleic acid). It is preferred according to the invention for a protein the recognition signal is an epitope and/or a paratope and/or a hapten and for a cDNA is a hybridization or binding region.

The marker sequences according to the invention are the subject matter of Table A and can be clearly identified by the respectively cited database entry (also by means of the Internet: http://www.ncbi.nlm.nih.gov/) (see in Table A: accession no. there).

According to the invention, the marker sequences also cover those modifications of the cDNA sequence and the corresponding amino acid sequence as chemical modification, such as citrullination, acetylation, phosphorylation, glycosylation or poly(A) strand and other modifications known to one skilled in the art.

In a further embodiment of the invention, partial sequences or fragments of the marker sequences according to the invention are likewise covered. In particular those partial sequences that have an identity of 95%, 90%, in particular 80% or 70% with the marker sequences according to the invention.

In a further embodiment, the respective marker sequence can be represented in different quantities in one more regions on a solid support. This permits a variation of the sensitivity. The regions can have respectively a totality of marker sequences, i.e., a sufficient number of different marker sequences, in particular 2 to 5 or 10 or more and optionally more nucleic acids and/or proteins, in particular biomarkers. However, at least 96 to 25,000 (numerical) or more from different or identical marker sequences and further nucleic acids and/or proteins, in particular biomarkers are preferred. Furthermore preferred are more than 2, 5000, in particular preferred 10,000 or more different or identical marker sequences and optionally further nucleic acids and/or proteins, in particular biomarkers.

Another object of the invention relates to an arrangement of marker sequences containing at least one marker sequence of a cDNA selected from the group SEQ 1-395 or respectively a protein coding therefor. Preferably, the arrangement contains at least 2 to 5 or 10, preferably 30 to 50 marker sequences or 50 to 100 or more marker sequences. Within the scope of this invention, “arrangement” is synonymous with “array,” and if this “array” is used to identify substances on marker sequences, this is to be understood to be an “assay” or diagnostic device. In a preferred embodiment, the arrangement is designed such that the marker sequences represented on the arrangement are present in the form of a grid on a solid support. Furthermore, those arrangements are preferred that permit a high-density arrangement of protein binders and the marker sequences are spotted. Such high-density spotted arrangements are disclosed, for example, in WO 99/57311 and WO 99/57312 and can be used advantageously in a robot-supported automated high-throughput method.

Within the scope of this invention, however, the term “assay” or diagnostic device likewise comprises those embodiments of a device, such as ELISA (e.g., individual wells of a microtiter plate are coated with the marker sequences or combinations of marker sequences according to the invention, optionally applied in a robot-supported manner in the individual wells of the microtiter plate; examples are diagnostic ELISA kits by Phadia or “Searchlight” multiplex ELISA kits by Pierce/Thermo Fisher Scientific), bead-based assay (spectrally distinguishable bead populations are coated with marker sequences/combinations of marker sequences. The patient sample is incubated with this bead population and bound (auto) antibodies are detected by means of a further fluorescence-labeled secondary antibody/detection reagent via measurement of the fluorescence; i.e., Borrelia IgG kit or Athena Multilyte by Multimetrix), line assay (marker sequences according to the invention or combinations of marker sequences are immobilized on membranes in a robot-supported manner, which are examined/incubated with the patient sample; example “Euroline” by Euroimmun AG), Western Blot (example “Euroline-WB” by Euroimmun AG), immunochromatographic methods (e.g., lateral flow immunoassays; marker sequences/combinations of marker sequences are immobilized on test strips (membranes, U.S. Pat. No. 5,714,389 and the like); example “One Step HBsAg” test device by Acon Laboratories) or similar immunological single or multiplex detection measures.

The marker sequences of the arrangement are fixed on a solid support, but preferably spotted or immobilized even printed on, i.e. applied in a reproducible manner. One or more marker sequences can be present multiple times in the totality of all marker sequences and present in different quantities based on one spot. Furthermore, the marker sequences can be standardized on the solid support (i.e., by means of serial dilution series of, e.g., human globulins as internal calibrators for data normalization and quantitative evaluation).

The invention therefore relates to an assay or a protein biochip comprising an arrangement containing marker sequences according to the invention.

In a further embodiment, the marker sequences are present as clones. Clones of this type can be obtained, for example, by means of a cDNA expression library according to the invention (Büssow et al. 1998 (supra)). In a preferred embodiment, such expression libraries containing clones are obtained using expression vectors from a cDNA expression library comprising the cDNA marker sequences. These expression vectors preferably contain inducible promoters. The induction of the expression can be carried out, e.g., by means of an inductor, such as IPTG. Suitable expression vectors are described in Terpe et al. (Terpe T Appl Microbiol Biotechnol. 2003 January; 60(5): 523-33).

One skilled in the art is familiar with expression libraries, they can be produced according to standard works, such as Sambrook et al, “Molecular Cloning, A laboratory handbook, 2nd edition (1989), CSH press, Cold Spring Harbor, N.Y. Expression libraries are also preferred which are tissue-specific (e.g., human tissue, in particular human organs). Furthermore included according to the invention are expression libraries that can be obtained by exon-trapping. A synonym for expression library is expression bank.

Also preferred are protein biochips or corresponding expression libraries that do not exhibit any redundancy (so-called: Uniclone® library) and that may be produced, for example, according to the teachings of WO 99/57311 and WO 99/57312. These preferred Uniclone libraries have a high portion of non-defective fully expressed proteins of a cDNA expression library.

Within the context of this invention, the clones can also be, but not limited to, transformed bacteria, recombinant phages or transformed cells from mammals, insects, fungi, yeasts or plants.

The clones are fixed, spotted or immobilized on a solid support.

The invention therefore relates to an arrangement wherein the marker sequences are present as clones.

Additionally, the marker sequences can be present in the respective form of a fusion protein, which contains, for example, at least one affinity epitope or tag. The tag may be one such as contains c-myc, his tag, arg tag, FLAG, alkaline phosphatase, VS tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase or lacZ.

A marker sequence can also be composed of several individual marker sequences. This can comprise the cloning of individual fragments to form a large common fragment and the expression of this combined fragment.

In all of the embodiments, the term “solid support” covers embodiments such as a filter, a membrane, a magnetic or fluorophore-labeled bead, a silica wafer, glass, metal, ceramics, plastics, a chip, a target for mass spectrometry or a matrix. However, a filter is preferred according to the invention.

As a filter, furthermore PVDF, nitrocellulose or nylon is preferred (e.g., Immobilon P Millipore, Protran Whatman, Hybond N+Amersham).

In another preferred embodiment of the arrangement according to the invention, the arrangement corresponds to a grid with the dimensions of a microtiter plate (8-12 wells strips, 96 wells, 384 wells or more), a silica wafer, a chip, a target for mass spectrometry, or a matrix.

In a further embodiment, the invention relates to an assay or a protein biochip for identifying and characterizing a substance for multiple sclerosis, characterized in that an arrangement or assay according to the invention is a.) brought into contact with at least one substance to be tested and b.) a binding success is detected.

Furthermore, the invention relates to a method for identifying and characterizing a substance for multiple sclerosis, characterized in that an arrangement or assay according to the invention is a.) brought into contact with at least one substance to be tested and b.) a binding success is detected.

The substance to be tested can be any native or non-native biomolecule, a synthetic chemical molecule, a mixture or a substance library.

After the substance to be tested contacts a marker sequence, the binding success is evaluated, which, for example, is carried out using commercially available image analyzing software (GenePix Pro (Axon Laboratories), Aida (Ray test), ScanArray (Packard Bioscience).

The visualization of protein-protein interactions according to the invention (e.g., protein on marker sequence, as antigen/antibody) or corresponding “means for detecting the binding success” can be performed, for example, using fluorescence labeling, biotinylation, radioisotope labeling or colloid gold or latex particle labeling in the usual way. A detection of bound antibodies is carried out with the aid of secondary antibodies, which are labeled with commercially available reporter molecules (e.g., Cy, Alexa, Dyomics, FITC or similar fluorescent dyes, colloidal gold or latex particles), or with reporter enzymes, such as alkaline phosphatase, horseradish peroxidase, etc., and the corresponding colorimetric, fluorescent or chemiluminescent substrates. Readout is conducted, e.g., using a microarray laser scanner, a CCD camera or visually.

In a further embodiment, the invention relates to a drug/active substance or prodrug developed for multiple sclerosis and obtainable through the use of the assay or protein biochip according to the invention.

The invention therefore likewise relates to the use of an arrangement according to the invention or an assay for screening active substances for multiple sclerosis.

In a further embodiment, the invention therefore likewise relates to a target for the treatment and therapy of multiple sclerosis respectively selected from the group SEQ 1-395 or a protein respectively coding therefor.

In a further embodiment, the invention likewise relates to the use of the marker sequences according to the invention, preferably in the form of an arrangement, as an affinity material for carrying out an apheresis or in the broadest sense a blood lavage, wherein substances from body fluids of a patient with multiple sclerosis, such as blood or plasma, bind to the marker sequences according to the invention and consequently can be selectively withdrawn from the body fluid.

### EXAMPLES AND FIGURES

Ten or more patient samples were individually screened against a cDNA expression library. The multiple sclerosis-specific expression clones were determined through a comparison with ten or more healthy samples. The identity of the marker sequences was determined by DNA sequencing.

FIG. 1 shows the differential screening between two protein biochips from respectively one cDNA expression bank of a patient and a healthy test subject. The differential clones are detected by means of fluorescent labeling and evaluated by means of bioinformatics.

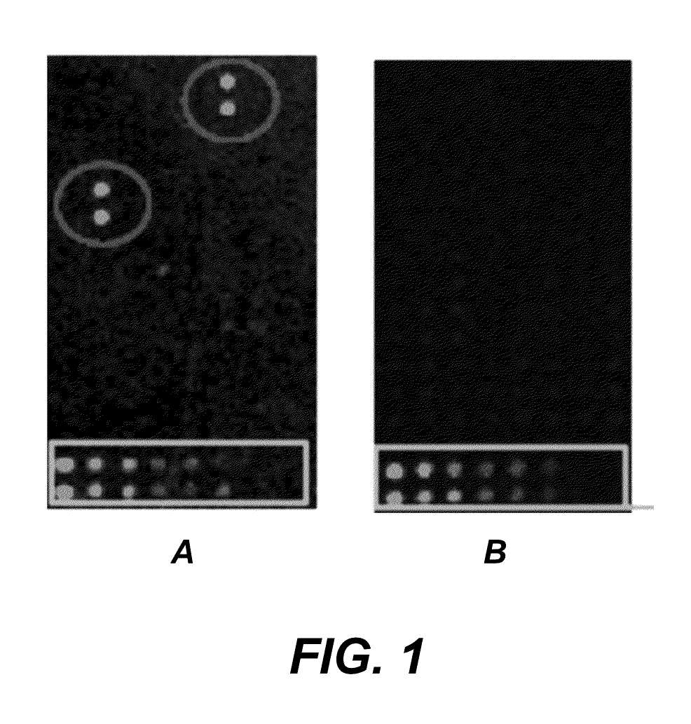

TABLE A

| | | | |
|---|---|---|---|
| 76 | 00800_544_G03 | NT_024000 | Homo sapiens chromosome 9 genomic contig, reference assembly |
| 77 | 00800_545_A07 | NM_004380 | Homo sapiens CREB binding protein (Rubinstein-Taybi syndrome) (CREBBP), mRNA |
| 78 | 00800_545_C01 | NT_010393 | Homo sapiens chromosome 16 genomic contig, reference assembly |
| 79 | 00800_545_J02 | NM_138559 | Homo sapiens B-cell CLL/lymphoma 11A (zinc finger protein) (BCL11A), |
| 80 | 00800_545_O21 | | transcript variant 3, mRNA |
| 81 | 00800_546_O21 | NM_006924 | Homo sapiens splicing factor, arginine/serine-rich 1 (splicing factor 2, |
| | | | alternate splicing factor) (SFRS1), mRNA |
| 82 | 00800_548_E20 | NM_002813 | Homo sapiens proteasome (prosome, macropain) 26S subunit, non-ATPase, 9 |
| | | | (PSMD9), mRNA |
| 83 | 00800_548_F15 | NM_024832 | Homo sapiens Ras and Rab interactor 3 (RIN3), mRNA |
| 84 | 00800_548_P22 | NM_022156 | Homo sapiens dihydrouridine synthase 1-like (S. cerevisiae) (DUS1L), mRNA |
| 85 | 00800_549_F07 | NM_014497 | Homo sapiens zinc finger protein 638 (ZNF638), transcript variant 1, mRNA |
| 86 | 00800_550_A02 | NM_016406 | Homo sapiens ubiquitin-fold modifier conjugating enzyme 1 (UFC1), mRNA |
| 87 | 00800_551_L21 | NM_005861 | Homo sapiens STIP1 homology and U-box containing protein 1 (STUB1), mRNA |
| 88 | 00800_551_M08 | NM_001569 | Homo sapiens interleukin-1 receptor-associated kinase 1 (IRAK1), transcript |
| | | | variant 1, mRNA |
| 89 | 00800_552_D16 | NM_012398 | Homo sapiens phosphatidylinositol-4-phosphate 5-kinase, type I, gamma |
| | | | (PIP5K1C), mRNA |
| 90 | 00800_552_E08 | NT_037887 | Homo sapiens chromosome 16 genomic contig, reference assembly |
| 91 | 00800_552_K06 | | |
| 92 | 00800_554_G09 | NM_006185 | Homo sapiens nuclear mitotic apparatus protein 1 (NUMA1), mRNA |
| 93 | 00800_554_P20 | NW_926018 | Homo sapiens chromosome 16 genomic contig, alternate assembly |
| | | | (based on Celera assembly) |
| 94 | 00800_556_D10 | NM_001005751 | Homo sapiens similar to KIAA0592 protein (LOC387680), mRNA |
| 95 | 00800_557_I07 | NM_024040 | Homo sapiens CUE domain containing 2 (CUEDC2), mRNA |
| 96 | 00800_558_M02 | NM_005861 | Homo sapiens STIP1 homology and U-box containing protein 1 (STUB1), mRNA |
| 97 | 00800_559_B12 | NM_022370 | Homo sapiens roundabout, axon guidance receptor, homolog 3 (Drosophila) |
| | | | (ROBO3), mRNA |
| 98 | 00800_562_H23 | NT_010641 | Homo sapiens chromosome 17 genomic contig, reference assembly |
| 99 | 00800_563_H18 | NW_926018 | Homo sapiens chromosome 16 genomic contig, alternate assembly |
| | | | (based on Celera assembly) |
| 100 | 00800_568_M18 | NM_002383 | Homo sapiens MYC-associated zinc finger protein |
| | | | (purine-binding transcription factor) (MAZ), mRNA |
| 101 | 00800_569_P20 | NM_019116 | Homo sapiens ubiquitin-binding protein homolog (UBPH), mRNA |
| 102 | 00800_573_M18 | NM_016162 | Homo sapiens inhibitor of growth family, member 4 (ING4), mRNA |
| 103 | 00800_573_P23 | NM_016474 | Homo sapiens chromosome 3 open reading frame 19 (C3orf19), mRNA |
| 104 | 00800_574_H11 | NM_182471 | Homo sapiens pyruvate kinase, muscle (PKM2), transcript variant 3, mRNA |
| 105 | 00800_577_J03 | XM_001126211 | PREDICTED: Homo sapiens similar to deoxythymidylate kinase |
| | | | (thymidylate kinase), transcript variant 4 (LOC727761), mRNA |
| 106 | 00800_578_A04 | XM_001132864 | PREDICTED: Homo sapiens huntingtin interacting protein 1 related |
| | | | (HIP1R), mRNA |
| 107 | 00800_578_I13 | NM_022762 | Homo sapiens required for melotic nuclear division 5 homolog B |
| | | | (S. cerevisiae) (RMND5B), mRNA |
| 108 | 00800_578_I17 | NT_023666 | Homo sapiens chromosome 8 genomic contig, reference assembly |
| 109 | 00800_578_I19 | NM_016162 | Homo sapiens inhibitor of growth family, member 4 (ING4), mRNA |
| 110 | 00800_578_L14 | NM_016162 | Homo sapiens inhibitor of growth family, member 4 (ING4), mRNA |
| 111 | 00800_578_L20 | NM_032514 | Homo sapiens microtubule-associated protein 1 light chain 3 alpha (MAP1LC3A), |
| | | | transcript variant 1, mRNA |
| 112 | 00800_578_N23 | NM_030795 | Homo sapiens stathmin-like 4 (STMN4), mRNA |
| 113 | 00800_578_P10 | NM_024040 | Homo sapiens CUE domain containing 2 (CUEDC2), mRNA |
| 114 | 00800_578_P05 | NM_002383 | Homo sapiens MYC-associated zinc finger protein |
| | | | (purine-binding transcription factor) (MAZ), mRNA |
| 115 | 00800_579_A01 | NM_022898 | Homo sapiens B-cell CLL/lymphoma 11B (zinc finger protein) (BCL11B), |
| | | | transcript variant 2, mRNA |
| 116 | 00800_579_P10 | NM_001018097 | Homo sapiens GRINL1A combined protein (Gcom1), transcript variant 9, mRNA |
| 117 | 00800_580_A12 | NM_162705 | Homo sapiens polymerase (RNA) I polypeptide D, 16 kDa (POLR1D), transcript |
| | | | variant 2, mRNA |
| 118 | 00800_581_K11 | NM_002825 | Homo sapiens pleiotrophin (heparin binding growth factor 8, neurite growth- |
| | | | promoting factor 1) (PTN), mRNA |
| 119 | 00800_581_L24 | NT_011520 | Homo sapiens chromosome 22 genomic contig, reference assembly |
| 120 | 00800_582_E09 | NT_037887 | Homo sapiens chromosome 16 genomic contig, reference assembly |
| 121 | 00800_582_I09 | NM_002032 | Homo sapiens ferritin, heavy polypeptide 1 (FTH1), mRNA |
| 122 | 00800_582_K06 | NM_001262 | Homo sapiens cyclin-dependent kinase inhibitor 2C (p18, inhibits CDK4) |
| | | | (CDKN2C), transcript variant 1, mRNA |
| 123 | 00800_582_M24 | NM_181697 | Homo sapiens peroxiredoxin 1 (PRDX1), transcript variant 3, mRNA |
| 124 | 00800_584_G07 | NM_014944 | Homo sapiens calsyntenin 1 (CLSTN1), transcript variant 2, mRNA |
| 125 | 00800_584_K08 | NM_002013 | Homo sapiens FK506 binding protein 3, 25 kDa (FKBP3), mRNA |
| 126 | 00800_584_M24 | NM_005626 | Homo sapiens splicing factor, arginine/serine-rich 4 (SFRS4), mRNA |
| 127 | 00800_585_P03 | NM_001005751 | Homo sapiens similar to KIAA0592 protein (LOC387680), mRNA |
| 128 | 00800_586_N14 | NM_007278 | Homo sapiens GABA(A) receptor-associated protein (GABARAP), mRNA |
| 129 | 00800_589_A19 | NM_003434 | Homo sapiens zinc finger protein 133 (ZNF133), mRNA |
| 130 | 00800_589_A21 | NM_003434 | Homo sapiens zinc finger protein 133 (ZNF133), mRNA |
| 131 | 00800_589_A07 | NM_015140 | Homo sapiens tubulin tyroslne ligase-like family, member 12 (TTLL12), mRNA |
| 132 | 00800_589_F10 | NM_012398 | Homo sapiens phosphatidylinositol-4-phosphate 5-kinase, type I, gamma (PIP5K1C), |
| | | | mRNA |
| 133 | 00800_589_I02 | NM_018683 | Homo sapiens zinc finger protein 313 (ZNF313), mRNA |
| 134 | 00800_589_M21 | NM_153849 | Homo sapiens tropomyosin 3 (TPM3), transcript variant 2, mRNA |
| 135 | 00800_590_A22 | NM_024040 | Homo sapiens CUE domain containing 2 (CUEDC2), mRNA |
| 136 | 00800_590_A24 | NM_024040 | Homo sapiens CUE domain containing 2 (CUEDC2), mRNA |
| 137 | 00800_590_B11 | NM_020967 | Homo sapiens nuclear receptor coactivator 5 (NCOA5), mRNA |
| 138 | 00800_590_C18 | NM_018406 | Homo sapiens ubiquitin-fold modifier conjugating enzyme 1 (UFC1), mRNA |
| 139 | 00800_591_H12 | NM_001010926 | Homo sapiens hairy and enhancer of split 5 (Drosophila) (HES5), mRNA |
| 140 | 00800_591_K11 | NM_003926 | Homo sapiens methyl-CpG binding domain protein 3 (MBD3), mRNA |
| 141 | 00800_591_M16 | NM_003130 | Homo sapiens sorcin (SRI), transcript variant 1, mRNA |
| 142 | 00800_592_H23 | NM_144999 | Homo sapiens leucine rich repeat containing 45 (LRRC45), mRNA |
| 143 | 00800_592_I16 | NM_002475 | Homo sapiens myosin, light chain 6B, alkali, smooth muscle and non-muscle |
| | | | (MYL6B), mRNA |
| 144 | 00800_592_K05 | NM_000477 | Homo sapiens albumin (ALB), mRNA |
| 145 | 00800_594_I15 | NM_016300 | Homo sapiens cyclic AMP-regulated phosphoprotein, 21 kD (ARPP-21), |
| | | | transcript variant 1, mRNA |
| 146 | 00800_594_M05 | NM_022839 | Homo sapiens mitochondrial ribosomal protein S11 (MRPS11), nuclear gene encoding |
| | | | mitochondrial protein, transcript variant 1, mRNA |
| 147 | 00800_595_J02 | NM_001281 | Homo sapiens cytoskeleton associated protein 1 (CKAP1), mRNA |
| 148 | 00800_595_K14 | NM_182471 | Homo sapiens pyruvate kinase, muscle (PKM2), transcript variant 3, mRNA |
| 149 | 00800_595_P16 | NM_005861 | Homo sapiens STIP1 homology and U-box containing protein 1 (STUB1), mRNA |
| 150 | 00800_596_A10 | NM_006086 | Homo sapiens tubulin, beta 3 (TUBB3), mRNA |
| 151 | 00800_596_C04 | NM_015894 | Homo sapiens stathmin-like 3 (STMN3), mRNA |
| 152 | 00800_596_D09 | NM_006086 | Homo sapiens tubulin, beta 3 (TUBB3), mRNA |
| 153 | 00800_596_E08 | NM_013442 | Homo sapiens stomatin (EPB72)-like 2 (STOML2), mRNA |
| 154 | 00800_596_N16 | XM_001126014 | PREDICTED: Homo sapiens similar to Cyclin-L2 (Paneth cell-enhanced expression |
| | | | protein), transcript variant 1 (LOC727877), mRNA |
| 155 | 00800_596_N21 | NM_018434 | Homo sapiens ring finger protein 130 (RNF130), mRNA |
| 156 | 00800_597_D15 | NM_024671 | Homo sapiens zinc finger protein 768 (ZNF768), mRNA |
| 167 | 00800_598_H13 | | |
| 158 | 00800_598_J16 | | |
| 159 | 00800_599_C24 | NM_001321 | Homo sapiens cysteine and glycine-rich protein 2 (CSRP2), mRNA |
| 160 | 00800_599_P14 | NM_182923 | Homo sapiens kinesin 2 (KNS2), transcript variant 2, mRNA |
| 161 | 00800_600_E13 | NM_002475 | Homo sapiens myosin, light chain 68, alkali, smooth muscle and non-muscle |
| | | | (MYL6B), mRNA |
| 162 | 00800_600_J10 | | |
| 163 | 00800_600_L06 | NM_001040134 | Homo sapiens paralemmin (PALM), transcript variant 2, mRNA |
| 164 | 00800_600_P10 | | |
| 165 | 00800_601_C10 | | |
| 166 | 00800_601_D06 | NM_005861 | Homo sapiens STIP1 homology and U-box containing protein 1 (STUB1), mRNA |
| 167 | 00800_602_B15 | | |
| 168 | 00800_602_M24 | NT_010194 | Homo sapiens chromosome 15 genomic contig, reference assembly |
| 169 | 00800_603_I12 | NM_014497 | Homo sapiens zinc finger protein 638 (ZNF638), transcript variant 1, mRNA |
| 170 | 09016_002_E20 | NM_004960 | Homo sapiens fusion (involved in t(12; 16) in malignant liposarcoma)(FUS) |
| 171 | 09016_002_J18 | NM_000973 | Homo sapiens ribosomal protein L8 (RPL8) |
| 172 | 09016_005_A20 | NM_004499 | Homo sapiens heterogeneous nuclear ribonucleoprotein A/B (HNRPAB) |
| 173 | 09016_005_H19 | NM_004593 | Homo sapiens splicing factor, arginine/serine-rich 10 (SFRS10) |
| 174 | 09016_005_O21 | NM_153649 | Homo sapiens tropomyosin 3 (TPM3) |
| 175 | 09016_008_B04 | NM_004494 | omo sapiens hepatoma-derived growth factor (high-mobility group protein 1-like) |
| | | | (HDGF) |
| 176 | 09016_009_H22 | NM_138400 | Homo sapiens nucleolar protein with MIF4G domain 1 (NOM1) |
| 177 | 09016_009_O04 | NM_002473 | Homo sapiens myosin, heavy chain 9, non-muscle (MYH9) |
| 178 | 09016_013_E04 | NM_012186 | Homo sapiens forkhead box E3 (FOXE3) |
| 179 | 09016_016_M20 | NM_020713 | Homo sapiens KIAA1196 protein (GM632) |
| 180 | 09016_017_F21 | NM_002473 | Homo sapiens myosin, heavy chain 9, non-muscle (MYH9) |
| 181 | 09016_018_O16 | NM_003475 | Homo sapiens Ras association (RalGDS/AF-6) domain family 7 (RASSF7) |
| 182 | 09016_020_A08 | NM_002819 | Homo sapiens polypyrimidine tract binding protein 1 (PTBP1) |
| 183 | 09016_020_F03 | NM_002819 | Homo sapiens polypyrimidine tract binding protein 1 (PTBP1) |
| 184 | 09016_020_F19 | NM_004559 | Homo sapiens Y box binding protein 1 (YBX1) |
| 185 | 09016_021_O08 | NM_004559 | Homo sapiens Y box binding protein 1 (YBX1) |
| 186 | 09016_022_D05 | NM_004494 | Homo sapiens hepatoma-derived growth factor (high-mobility group protein 1-like) |
| | | | (HDGF) |
| 187 | 09016_023_A16 | NM_004559 | Homo sapiens Y box binding protein 1 (YBX1) |
| 188 | 09016_023_O09 | NM_138400 | Homo sapiens nucleolar protein with MIF4G domain 1 (NOM1) |
| 189 | 09016_024_M04 | NM_004559 | Homo sapiens Y box binding protein 1 (YBX1) |
| 190 | 09016_028_M09 | NM_001034025 | Homo sapiens endoplasmic reticulum protein 29 (ERP29) |
| 191 | 09016_031_C13 | NM_033112 | Homo sapiens chromosome 6 open reading frame 153 (C6orf153) |
| 192 | 09016_031_E24 | NM_002819 | Homo sapiens polypyrimidine tract binding protein 1 (PTBP1) |
| 193 | 09016_034_D03 | NM_004593 | omo sapiens splicing factor, arginine/serine-rich 10 (SFRS10) |
| 194 | 09017_003_E21 | NM_021009 | Homo sapiens ubiquitin C (UBC) |
| 195 | 09017_004_G23 | NM_001008657 | Homo sapiens Treacher Collins-Franceschetti syndrome 1 (TCOF1) |
| 196 | 09017_012_B14 | NM_016257 | Homo sapiens hippocalcin like 4 (HPCAL4) |
| 197 | 09017_015_O07 | NM_018690 | Homo sapiens apolipoprotein B48 receptor (APOB48R) |
| 198 | 09017_016_N21 | NM_004559 | Homo sapiens Y box binding protein 1 (YBX1) |
| 199 | 09017_019_H17 | NM_079421 | Homo sapiens cyclin-dependent kinase inhibitor 2D (p19, Inhibits CDK4) |
| | | | (CDKN2D) |
| 200 | 09017_020_C19 | NM_004494 | Homo sapiens hepatoma-derived growth factor (high-mobility group protein |
| | | | 1-like) (HDGF) |
| 201 | 09017_021_H07 | NM_001034025 | Homo sapiens endoplasmic reticulum protein 29 (ERP29) |
| 202 | 09017_022_E05 | NM_001012 | Homo sapiens ribosomal protein S8 (RPS8) |
| 203 | 09017_022_G11 | NM_014593 | Homo sapiens CXXC finger 1 (PHD domain) (CXXC1), mRNA |
| 204 | 09017_022_N22 | NM_001010850 | Homo sapiens fusion (involved in t(12; 16) in malignant liposarcoma) |
| | | | (FUS), transcript variant 2, mRNA |
| 205 | 09017_027_B02 | NM_007158 | Homo sapiens cold shock domain containing E1, RNA-binding (CSDE1), |
| | | | transcript variant 2, mRNA |
| 206 | 09017_030_I14 | NM_001312 | Homo sapiens cysteine-rich protein 2 (CRIP2), mRNA |
| 207 | 09017_030_M06 | NM_032251 | Homo sapiens coiled-coil domain containing 88 (CCDC88), mRNA |
| 208 | 09017_032_D09 | NM_002228 | Homo sapiens jun oncogene (JUN), mRNA |
| 209 | 09017_032_G02 | NM_005354 | Homo sapiens jun D proto-oncogene (JUND), mRNA |
| 210 | 09017_032_O04 | NM_022898 | Homo sapiens B-cell CLL/lymphoma 11B (zinc finger protein) (BCL11B), |
| | | | transcript variant 2, mRNA |
| 211 | 09017_034_L19 | NM_005157 | Homo sapiens v-abl Abelson murine leukemia viral oncogens homolog 1 |
| | | | (ABL1), transcript variant a, mRNA |
| 212 | 09017_039_I18 | NM_002383 | Homo sapiens MYC-associated zinc finger protein (purine-binding |
| | | | transcription factor) (MAZ), mRNA |
| 213 | 09017_042_D04 | NM_002473 | Homo sapiens myosin, heavy chain 9, non-muscle (MYH9), mRNA |
| 214 | MyelinBasicProteinAnti- | | |
| | genfromHumanbrain | | |
| 215 | 00800_505_F06 | NM_013235 | Homo sapiens ribonuclease III, nuclear (RNASEN), mRNA |
| 216 | 00800_505_I09 | NM_001636 | Homo sapiens solute carrier family 25 (mitochondrial carrier; adenine |
| | | | nucleotide translocator), member 6 (SLC25A6), mRNA |
| 217 | 00800_506_M11 | NT_021937 | Homo sapiens chromosome 1 genomic contig, reference assembly |
| 218 | 00800_507_M15 | NM_000127 | Homo sapiens exostoses (multiple) 1 (EXT1), mRNA |
| 219 | 00800_508_I24 | NM_004854 | Homo sapiens carbohydrate sulfotransferase 10 (CHST10), mRNA |
| 220 | 00800_510_B13 | | |
| 221 | 00800_510_J11 | NW_926918 | Homo sapiens chromosome 17 genomic contig, alternate assembly |
| | | | (based on Celera assembly) |
| 222 | 00800_512_C01 | | |
| 223 | 00800_512_O19 | NM_006160 | Homo sapiens neurogenic differentiation 2 (NEUROD2), mRNA |
| 224 | 00800_513_E09 | | |
| 225 | 00800_513_M06 | NM_005937 | Homo sapiens myeloid/lymphoid or mixed-lineage leukemia (trithorax |
| | | | homolog, Drosophila); translocated to, 6 (MLLT6), mRNA |
| 226 | 00800_513_N07 | NT_011630 | Homo sapiens chromosome X genomic contig, reference assembly |
| 227 | 00800_514_A15 | NM_003824 | Homo sapiens Fas (TNFRSF6)-associated via death domain (FADD), mRNA |
| 228 | 00800_514_H03 | XM_001123454 | PREDICTED: Homo sapiens GIY-YIG domain containing 2, transcript |
| | | | variant 1 (GIYD2), mRNA |
| 229 | 00800_515_C16 | | |
| 230 | 00800_515_C22 | | |
| 231 | 00800_518_K18 | NM_014851 | Homo sapiens kelch-like 21 (Drosophila) (KLHL21), mRNA |
| 232 | 00800_518_O10 | NM_004838 | Homo sapiens homer homolog 3 (Drosophila) (HOMER3), mRNA |
| 233 | 00800_519_A23 | | |
| 234 | 00800_523_B20 | NT_009775 | Homo sapiens chromosome 12 genomic contig, reference assembly |
| 235 | 00800_523_B06 | NM_198318 | Homo sapiens protein arginine methyltransferase 1 (PRMT1), |
| | | | transcript variant 3, mRNA |
| 236 | 00800_523_I13 | NM_032333 | Homo sapiens chromosome 10 open reading frame 58 (C10orf58), mRNA |
| 237 | 00800_523_K19 | | |
| 238 | 00800_523_P18 | NM_144576 | Homo sapiens coenzyme Q10 homolog A (S. cerevisiae) (COQ10A), mRNA |
| 239 | 00800_524_A06 | XM_001131713 | PREDICTED: Homo sapiens similar to HLA class I histocompatibility antigen, |
| | | | B-18 alpha chain precursor (MHC class I antigen B*18) (LOC730410), mRNA |
| 240 | 00800_524_C12 | | |
| 241 | 00800_524_D10 | NM_030815 | Homo sapiens p53 and DNA damage regulated 1 (PDRG1), mRNA |
| 242 | 00800_524_E24 | NM_016162 | Homo sapiens inhibitor of growth family, member 4 (ING4), mRNA |
| 243 | 00800_524_I01 | NM_021727 | Homo sapiens fatty acid desaturase 3 (FADS3), mRNA |
| 244 | 00800_524_K14 | | |
| 245 | 00800_524_N04 | NM_002856 | Homo sapiens poliovirus receptor-related 2 (herpesvirus entry mediator B) |
| | | | (PVRL2), mRNA |
| 246 | 00800_525_M19 | NM_004095 | Homo sapiens eukaryotic translation initiation factor-4E binding protein 1 |
| | | | (EIF4EBP1), mRNA |
| 247 | 00800_526_C01 | | |
| 248 | 00800_526_D09 | NM_006644 | Homo sapiens heat shock 105 kDa/110 kDa protein 1 (HSPH1), mRNA |
| 249 | 00800_526_G11 | NT_008413 | Homo sapiens chromosome 9 genomic contig, reference assembly |
| 250 | 00800_528_A16 | | |
| 251 | 00800_528_D24 | NM_052880 | Homo sapiens HGFL gene (MGC17330), mRNA |
| 252 | 00800_528_F07 | NM_001015885 | Homo sapiens RAE1 RNA export 1 homolog (S. pombe) (RAE1), transcript |
| | | | variant 2, mRNA |
| 253 | 00800_528_H06 | NM_031157 | Homo sapiens heterogeneous nuclear ribonucleoprotein A1 (HNRPA1), transcript |
| | | | variant 2, mRNA |
| 254 | 00800_528_M23 | NM_001008709 | Homo sapiens protein phosphatase 1, catalytic subunit alpha isoform (PPP1CA), |
| | | | transcript variant 3, mRNA |
| 255 | 00800_528_M05 | NM_001940 | Homo sapiens atrophin 1 (ATN1), transcript variant 2, mRNA |
| 256 | 00800_529_F03 | XM_001129992 | PREDICTED: Homo sapiens plasticity-related gene 2 (PRG2), mRNA |
| 257 | 00800_529_M18 | NM_030795 | Homo sapiens stathmin-like 4 (STMN4), mRNA |
| 258 | 00800_529_M22 | NM_001983 | Homo sapiens excision repair cross-complementing rodent repair deficiency, |
| | | | complementation group 1 (includes overlapping antisense sequence) (ERCC1), |
| | | | transcript variant 2, mRNA |
| 259 | 00800_529_O18 | NM_021149 | Homo sapiens coactosin-like 1 (Dictyostelium) (COTL1), mRNA |
| 260 | 00800_530_E13 | NM_002095 | Homo sapiens general transcription factor IIE, polypeptide 2, beta 34 kDa |
| | | | (GTF2E2), mRNA |
| 261 | 00800_530_J19 | NM_021149 | Homo sapiens coactosin-like 1 (Dictyostelium) (COTL1), mRNA |
| 262 | 00800_532_A06 | | |
| 263 | 00800_532_L19 | NM_012088 | Homo sapiens 6-phosphogluconolactonase (PGLS), mRNA |
| 264 | 00800_533_F22 | NM_020710 | Homo sapiens leucine rich repeat containing 47 (LRRC47), mRNA |
| 265 | 00800_533_G14 | NM_017542 | Homo sapiens pogo transposable element with KRAB domain (POGK), mRNA |
| 266 | 00800_534_C05 | | |
| 267 | 00800_536_F02 | NM_002660 | Homo sapiens phospholipase C, gamma 1 (PLCG1), transcript variant 1, mRNA |
| 268 | 00800_539_P13 | | |
| 269 | 00800_540_H17 | | |
| 270 | 00800_540_K16 | | |
| 271 | 00800_543_P22 | | |
| 272 | 00800_546_A20 | NM_012088 | Homo sapiens 6-phosphogluconolactonase (PGLS), mRNA |
| 273 | 00800_546_I15 | NM_007118 | Homo sapiens triple functional domain (PTPRF interacting) (TRIO), mRNA |
| 274 | 00800_548_A24 | NM_173551 | Homo sapiens ankyrin repeat and sterile alpha motif domain containing 6 |
| | | | (ANKS6), mRNA |
| 275 | 00800_548_A05 | NM_001031696 | Homo sapiens phospholipase D family, member 3 (PLD3), transcript variant 1, mRNA |
| 276 | 00800_548_B04 | NM_016035 | Homo sapiens coenzyme Q4 homolog (S. cerevisiae) (COQ4), mRNA |
| 277 | 00800_548_J12 | NM_012398 | Homo sapiens phosphatidylinositol-4-phosphate 5-kinase, type I, gamma |
| | | | (PIP5K1C), mRNA |
| 278 | 00800_549_G07 | XM_941435 | PREDICTED: Homo sapiens hypothetical protein LOC146909, transcript variant 4 |
| | | | (LOC146909), mRNA |
| 279 | 00800_550_B21 | NM_005474 | Homo sapiens histone deacetylase 5 (HDAC5), transcript variant 1, mRNA |
| 280 | 00800_552_D02 | NM_004573 | Homo sapiens phospholipase C, beta 2 (PLCB2), mRNA |
| 281 | 00800_552_F09 | NM_006768 | Homo sapiens BRCA1 associated protein (BRAP), mRNA |
| 282 | 00800_552_P18 | NT_010641 | Homo sapiens chromosome 17 genomic contig, reference assembly |
| 283 | 00800_556_D05 | NM_006805 | Homo sapiens heterogeneous nuclear ribonucleoprotein A0 (HNRPA0), mRNA |
| 284 | 00800_557_C02 | | |
| 285 | 00800_558_A22 | | |
| 286 | 00800_559_O11 | NM_024083 | Homo sapiens alveolar soft part sarcoma chromosome region, candidate 1 |
| | | | (ASPSCR1), mRNA |
| 287 | 00800_560_K09 | | |
| 288 | 00800_562_M02 | | |
| 289 | 00800_563_J13 | NM_012088 | Homo sapiens 6-phosphogluconolactonase (PGLS), mRNA |
| 290 | 00800_565_C16 | NM_005573 | Homo sapiens lamin B1 (LMNB1), mRNA |
| 291 | 00800_565_H12 | NT_019197 | Homo sapiens chromosome 22 genomic contig, reference assembly |
| 292 | 00800_566_E12 | NM_032019 | Homo sapiens histone deacetylase 10 (HDAC10), mRNA |
| 293 | 00800_566_M21 | NW_927284 | Homo sapiens chromosome 19 genomic contig, alternate assembly |
| | | | (based on Celera assembly) |
| 294 | 00800_569_B07 | NM_003180 | Homo sapiens synaptotagmin V (SYT5), mRNA |
| 295 | 00800_569_D11 | NM_023009 | Homo sapiens MARCKS-like 1 (MARCKSL1), mRNA |
| 296 | 00800_569_H21 | NM_020710 | Homo sapiens leucine rich repeat containing 47 (LRRC47), mRNA |
| 297 | 00800_570_E19 | NT_011295 | Homo sapiens chromosome 19 genomic contig, reference assembly |
| 298 | 00800_570_H05 | NT_035014 | Homo sapiens chromosome 9 genomic contig, reference assembly |
| 299 | 00800_570_I09 | NM_016292 | Homo sapiens TNF receptor-associated protein 1 (TRAP1), mRNA |
| 300 | 00800_574_D01 | NM_030795 | Homo sapiens stathmin-like 4 (STMN4), mRNA |
| 301 | 00800_574_E19 | | |
| 302 | 00800_576_B07 | NM_016139 | Homo sapiens coiled-coil-helix-coiled-coil-helix domain containing 2 |
| | | | (CHCHD2), mRNA |
| 303 | 00800_577_C12 | NM_004295 | Homo sapiens TNF receptor-associated factor 4 (TRAF4), transcript variant 1, |
| | | | mRNA |
| 304 | 00800_577_J13 | NM_006374 | Homo sapiens serine/threonine kinase 25 (STE20 homolog, yeast) (STK25), mRNA |
| 305 | 00800_577_K10 | NM_021991 | Homo sapiens junction plakoglobin (JUP), transcript variant 2, mRNA |
| 306 | 00800_577_L03 | XM_001129992 | PREDICTED: Homo sapiens plasticity-related gene 2 (PRG2), mRNA |
| 307 | 00800_J77_O11 | NM_002825 | Homo sapiens pleiotrophin (heparin binding-growth factor 8, neurite growth- |
| | | | promoting factor 1) (PTN), mRNA |
| 308 | 00800_578_B01 | | |
| 309 | 00800_578_O13 | NM_004559 | Homo sapiens Y box binding protein 1 (YBX1), mRNA |
| 310 | 00800_578_O22 | | |
| 311 | 00800_579_G06 | NT_007933 | Homo sapiens chromosome 7 genomic contig, reference assembly |
| 312 | 00800_579_L22 | NM_021149 | Homo sapiens coactosin-like 1 (Dictyostelium) (COTL1), mRNA |
| 313 | 00800_579_O08 | | |
| 314 | 00800_579_P11 | NM_003131 | Homo sapiens serum response factor (c-fos serum response element-binding |
| | | | transcription factor) (SRF), mRNA |
| 315 | 00800_581_C14 | NM_001003801 | Homo sapiens SWI/SNF related, matrix associated, actin dependent regulator of |
| | | | chromatin, subfamily d, member 3 (SMARCD3), transcript variant 3, mRNA |
| 316 | 00800_581_G17 | NM_001003801 | Homo sapiens SWI/SNF related, matrix associated, actin dependent regulator of |
| | | | chromatin, subfamily d, member 3 (SMARCD3), transcript variant 3, mRNA |
| 317 | 00800_581_L04 | MM 031454 | Homo sapiens selenoprotein O (SELO), mRNA |
| 318 | 00800_582_B01 | | |
| 319 | 00800_582_H11 | NM_152411 | Homo sapiens zinc finger protein 786 (ZNF788), mRNA |
| 320 | 00800_582_K12 | NM_001013725 | Homo sapiens hypothetical gene supported by BC044942 (LOC441268), mRNA |
| 321 | 00800_582_L08 | NM_005736 | Homo sapiens ARP1 actin-related protein 1 homolog A, centractin alpha (yeast) |
| | | | (ACTR1A), mRNA |
| 322 | 00800_584_D17 | NM_002613 | Homo sapiens 3-phosphoinositide dependent protein kinase-1 (PDPK1), transcript |
| | | | variant 1, mRNA |
| 323 | 00800_584_H24 | NM_020799 | Homo sapiens STAM binding protein-like 1 (STAMBPL1), mRNA |
| 324 | 00800_584_H06 | NM_021149 | Homo sapiens coactosin-like 1 (Dictyostelium) (COTL1), mRNA |
| 325 | 00800_585_H10 | NM_001002261 | Homo sapiens zinc finger, FYVE domain containing 27 (ZFYVE27), transcript |
| | | | variant 1, mRNA |
| 326 | 00800_585_H18 | NT_023133 | Homo sapiens chromosome 5 genomic contig, reference assembly |
| 327 | 00800_586_O09 | | |
| 328 | 00800_588_P23 | XM_940198 | PREDICTED: Homo sapiens hypothetical LOC644096 (LOC644096), mRNA |
| 329 | 00800_589_L09 | NT_011109 | Homo sapiens chromosome 19 genomic contig, reference assembly |
| 330 | 00800_589_N14 | NM_002751 | Homo sapiens mitogen-activated protein kinase 11 (MAPK11), mRNA |
| 331 | 00800_590_C08 | NM_006035 | Homo sapiens CDC42 binding protein kinase beta (DMPK-like) (CDC42BPB), mRNA |
| 332 | 00800_590_H08 | NM_005707 | Homo sapiens programmed cell death 7 (PDCD7), mRNA |
| 333 | 00800_590_K13 | NM_012226 | Homo sapiens nucleotide binding protein 2 (MinD homolog, E. coli) (NUBP2), |
| | | | mRNA |
| 334 | 00800_590_L18 | NM_005726 | Homo sapiens Ts translation elongation factor, mitochondrial (TSFM), mRNA |
| 335 | 00800_591_G04 | NM_006640 | Homo sapiens septin 9 (SEPT9), mRNA |
| 336 | 00800_591_G07 | | |
| 337 | 00800_591_K22 | NM_001024807 | Homo sapiens amyloid beta (A4) precursor-like protein 1 (APLP1), transcript |
| | | | variant 1, mRNA |
| 338 | 00800_594_P22 | NM_030815 | Homo sapiens p53 and DNA damage regulated 1 (PDRG1), mRNA |
| 339 | 00800_595_L06 | NW_926561 | Homo sapiens chromosome 16 genomic contig, alternate assembly |
| | | | (based on Celera assembly) |
| 340 | 00800_596_C18 | NM_001009998 | Homo sapiens single stranded DNA binding protein 4 (SSBP4), transcript |
| | | | variant 2, mRNA |
| 341 | 00800_596_F15 | | |
| 342 | 00800_596_J07 | NM_001031684 | Homo sapiens splicing factor, arginine/serine-rich 7, 35 kDa (SFRS7), mRNA |
| 343 | 00800_597_L02 | NM_018083 | Homo sapiens zinc finger protein 358 (ZNF358), mRNA |
| 344 | 00800_597_O20 | NT_032977 | Homo sapiens chromosome 1 genomic contig, reference assembly |
| 345 | 00800_598_D04 | NM_003768 | Homo sapiens phosphoprotein enriched in astrocytes 15 (PEA15), mRNA |
| 346 | 00800_598_I01 | NM_030815 | Homo sapiens p53 and DNA damage regulated 1 (PDRG1), mRNA |
| 347 | 00800_598_I19 | NM_004559 | Homo sapiens Y box binding protein 1 (YBX1), mRNA |
| 348 | 00800_598_N16 | NM_004559 | Homo sapiens Y box binding protein 1 (YBX1), mRNA |
| 349 | 00800_600_O12 | NM_006513 | Homo sapiens seryl-tRNA synthetase (SARS), mRNA |
| 350 | 00800_601_A11 | NM_016162 | Homo sapiens inhibitor of growth family, member 4 (ING4), mRNA |
| 351 | 00800_601_B18 | NM_032333 | Homo sapiens chromosome 10 open reading frame 58 (C10orf58), mRNA |
| 352 | 00800_601_J21 | NW_926918 | Homo sapiens chromosome 17 genomic contig, alternate assembly |
| | | | (based on Celera assembly) |
| 353 | 00800_601_K12 | NM_003333 | Homo sapiens ubiquitin A-52 residue ribosomal protein fusion product 1 |
| | | | (UBA52), transcript variant 2, mRNA |
| 354 | 00800_633_J02 | | |
| 355 | 09016_001_K02 | NM_170708 | Homo sapiens lamin A/C (LMNA) |
| 356 | 09016_002_C13 | NM_004418 | Homo sapiens dual specificity phosphatase 2 (DUSP2 |
| 367 | 09016_003_I24 | NM_015190 | Homo sapiens DnaJ (Hsp40) homolog, subfamily C, member 9 (DNAJC9) |
| 358 | 09016_003_P06 | NM_002954 | Homo sapiens ribosomal protein S27a (RPS27A) |
| 359 | 09016_004_L21 | NM_015190 | Homo sapiens DnaJ (Hsp40) homolog, subfamily C, member 9 (DNAJC9) |
| 360 | 09016_005_O03 | NM_004418 | Homo sapiens dual specificity phosphatase 2 (DUSP2) |
| 361 | 09016_006_J18 | NM_006796 | Homo sapiens AFG3 ATPase family gene 3-like 2 (yeast) (AFG3L2) |
| 362 | 09016_006_P23 | NM_004418 | Homo sapiens dual specificity phosphatase 2 (DUSP2) |
| 363 | 09016_017_L21 | NM_004418 | Homo sapiens dual specificity phosphatase 2 (DUSP2) |
| 364 | 09016_019_C05 | NM_002473 | Homo sapiens myosin, heavy chain 9, non-muscle (MYH9) |
| 365 | 09016_021_O07 | NM_000386 | Homo sapiens bleomycin hydrolase (BLMH |
| 366 | 09016_024_G16 | NM_004418 | Homo sapiens dual specificity phosphatase 2 (DUSP2) |
| 367 | 09016_024_L06 | NM_005572 | Homo sapiens lamin A/C (LMNA), transcript variant 2 |
| 368 | 09016_025_E24 | NM_004418 | Homo sapiens dual specificity phosphatase 2 (DUSP2) |
| 369 | 09016_030_B07 | NM_004418 | Homo sapiens dual specificity phosphatase 2 (DUSP2) |
| 370 | 09016_030_K04 | NM_005572 | Homo sapiens lamin A/C (LMNA), transcript variant 2 |
| 371 | 09016_034_E04 | NM_015190 | Homo sapiens DnaJ (Hsp40) homolog, subfamily C, member 9 (DNAJC9) |
| 372 | 09017_001_B21 | NM_004418 | Homo sapiens dual specificity phosphatase 2 (DUSP2) |
| 373 | 09017_002_K04 | NM_004418 | Homo sapiens dual specificity phosphatase 2 (DUSP2) |
| 374 | 09017_002_P24 | NM_004418 | Homo sapiens dual specificity phosphatase 2 (DUSP2) |
| 375 | 09017_003_B05 | NM_004418 | Homo sapiens dual specificity phosphatase 2 (DUSP2) |
| 376 | 09017_008_D17 | NM_004418 | Homo sapiens dual specificity phosphatase 2 (DUSP2) |
| 377 | 09017_008_I02 | NM_004418 | Homo sapiens dual specificity phosphatase 2 (DUSP2) |
| 378 | 09017_014_I23 | NM_020529 | Homo sapiens nuclear factor of kappa light polypeptide gene enhancer |
| | | | in B-cells inhibitor, alpha (NFKBIA) |
| 379 | 09017_016_J17 | NM_004418 | Homo sapiens dual specificity phosphatase 2 (DUSP2) |
| 380 | 09017_018_J21 | NM_002954 | Homo sapiens ribosomal protein S27a (RPS27A) |
| 381 | 09017_019_E20 | NM_004418 | Homo sapiens dual specificity phosphatase 2 (DUSP2) |
| 382 | 09017_019_H05 | NM_015711 | Homo sapiens glioma tumor suppressor candidate region gene 1 |
| | | | (GLTSCR1), |
| 383 | 09017_019_L21 | | |
| 384 | 09017_020_C09 | NM_004418 | Homo sapiens dual specificity phosphatase 2 (DUSP2) |
| 385 | 09017_021_L03 | NM_006360 | Homo sapiens PCI domain containing 1 (herpesvirus entry mediator) (PCID1) |
| 386 | 09017_022_F16 | NM_012292 | Homo sapiens histocompatibility (minor) HA-1 (HMHA1) |
| 387 | 09017_023_E01 | NM_002954 | Homo sapiens ribosomal protein S27a (RPS27A), mRNA |
| 388 | 09017_024_A21 | NM_004418 | Homo sapiens dual specificity phosphatase 2 (DUSP2), mRNA |
| 389 | 09017_024_C07 | NM_004418 | Homo sapiens dual specificity phosphatase 2 (DUSP2), mRNA |
| 390 | 09017_024_K03 | NM_002954 | Homo sapiens ribosomal protein S27a (RPS27A), mRNA |
| 391 | 09017_025_K17 | NM_004418 | Homo sapiens dual specificity phosphatase 2 (DUSP2), mRNA |
| 392 | 09017_026_L23 | NM_001025598 | Homo sapiens Rho GTPase activating protein 30 (ARHGAP30), transcript |
| | | | variant 1, mRNA |
| 393 | 09017_032_C07 | NM_004418 | Homo sapiens dual specificity phosphatase 2 (DUSP2), mRNA |
| 394 | 09017_036_G03 | NM_002473 | Homo sapiens myosin, heavy chain 9, non-muscle (MYH9), mRNA |
| 395 | 09017_019_L21 | | |
| 396 | 00800_505_H09 | | |
| 397 | 00800_523_E11 | NM_020655 | Homo sapiens junctophilin 3 (JPH3), mRNA |
| 398 | 00800_523_E04 | NM_147158 | Homo sapiens opioid receptor, sigma 1 (OPRS1), transcript variant 3, mRNA |
| 399 | 00800_523_G05 | NM_002808 | Homo sapiens proteasome (prosome, macropain) 26S subunit, non-ATPase, 3 |
| | | | (PSMD3), mRNA |
| 400 | 00800_523_L04 | XM_939744 | PREDICTED: Homo sapiens similar to cytoplasmic beta-actin (LOC644961), mRNA |
| 401 | 00800_526_A09 | | |
| 402 | 00800_526_O19 | NM_178148 | Homo sapiens solute carrier family 35, member B2 (SLC35B2), mRNA |
| 403 | 00800_528_J11 | | |
| 404 | 00800_529_D17 | | |
| 405 | 00800_530_J04 | NM_014902 | Homo sapiens discs, large (Drosophila) homolog-associated |
| | | | protein 4 (DLGAP4), transcript variant 1, mRNA |
| 406 | 00800_534_K03 | NT_011109 | Homo sapiens chromosome 19 genomic contig, reference assembly |
| 407 | 00800_538_I10 | | |
| 408 | 00800_538_J05 | NT_113943 | Homo sapiens chromosome 17 genomic contig, reference assembly |
| 409 | 00800_550_C21 | NM_005474 | Homo sapiens histone deacetylase 5 (HDAC5), transcript variant 1, mRNA |
| 410 | 00800_554_C07 | | |
| 411 | 00800_554_M18 | NT_029419 | Homo sapiens chromosome 12 genomic contig, reference assembly |
| 412 | 00800_557_I16 | NM_005442 | Homo sapiens eomesodermin homolog (Xenopus laevis) |
| | | | (EOMES), mRNA |
| 413 | 00800_558_I15 | NT_005416 | Homo sapiens chromosome 2 genomic contig, reference assembly |
| 414 | 00800_564_O09 | | |
| 415 | 00800_572_J16 | NM_032014 | Homo sapiens mitochondrial ribosomal protein S24 (MRPS24), nuclear gene |
| | | | encoding mitochondrial protein, mRNA |
| 416 | 00800_574_H13 | NT_011109 | Homo sapiens chromosome 19 genomic contig, reference assembly |
| 417 | 00800_578_E04 | NM_002714 | Homo sapiens protein phosphatase 1, regulatory subunit 10 (PPP1R10), mRNA |
| 418 | 00800_579_P03 | | |
| 419 | 00800_580_P08 | NM_002383 | Homo sapiens MYC-associated zinc finger protein (purine-binding transcription |
| | | | factor) (MAZ), mRNA |
| 420 | 00800_581_D11 | | |
| 421 | 00800_581_J21 | | |
| 422 | 00800_582_O11 | | |
| 423 | 00800_582_P05 | NM_001005366 | Homo sapiens F-box and leucine-rich repeat protein 10 (FBXL10), transcript |
| | | | variant 2, mRNA |
| 424 | 00800_583_B14 | NM_005984 | Homo sapiens solute carrier family 25 (mitochondrial carrier; citrate transporter), |
| | | | member 1 (SLC25A1), mRNA |
| 425 | 00800_583_D23 | NM_016930 | Homo sapiens syntaxin 18 (STX18), mRNA |
| 426 | 00800_583_G13 | NM_005276 | Homo sapiens glycerol-3-phosphate dehydrogenase 1 (soluble) (GPD1), mRNA |
| 427 | 00800_583_K19 | NM_015980 | Homo sapiens HMP19 protein (HMP19), mRNA |
| 428 | 00800_583_O03 | NM_006009 | Homo sapiens tubulin, alpha 3 (TUBA3), mRNA |
| 429 | 00800_584_G03 | NT_026437 | Homo sapiens chromosome 14 genomic contig, reference assembly |
| 430 | 00800_584_O14 | NM_024671 | Homo sapiens zinc finger protein 768 (ZNF768), mRNA |
| 431 | 00800_585_K18 | NT_016354 | Homo sapiens chromosome 4 genomic contig, reference assembly |
| 432 | 00800_585_N24 | NM_014405 | Homo sapiens calcium channel, voltage-dependent, gamma subunit 4 (CACNG4), mRNA |
| 433 | 00800_586_A24 | NM_004209 | Homo sapiens synaptogyrin 3 (SYNGR3), mRNA |
| 434 | 00800_586_H17 | | |
| 435 | 00800_590_P01 | NM_016257 | Homo sapiens hippocalcin like 4 (HPCAL4), mRNA |
| 438 | 00800_591_C18 | NM_015125 | Homo sapiens capicua homolog (Drosophila) (CIC), mRNA |
| 437 | 00800_591_F21 | XM_926195 | PREDICTED: Homo sapiens similar to Myc-associated zinc ringer protein (MAZI) |
| | | | (Purine-binding transcription factor) (Pur-1) (ZF87) (ZIFB7) (LOC642773), mRNA |
| 438 | 00800_592_I20 | | |
| 439 | 00800_592_K06 | NM_001069 | Homo sapiens tubulin, beta 2A (TUBB2A), mRNA |
| 440 | 00800_595_A14 | NM_022823 | Homo sapiens fibronectin type III domain containing 4 (FNDC4), mRNA |
| 441 | 00800_595_H19 | NM_001005362 | Homo sapiens dynamin 2 (DNM2), transcript variant 4, mRNA |
| 442 | 00800_597_B24 | NM_017789 | Homo sapiens sema domain, immunoglobulin domain (Ig), transmembrane domain |
| | | | (TM) and short cytoplasmic domain, (semaphorin) 4C (SEMA4C), mRNA |
| 443 | 00800_597_C09 | NM_033647 | Homo sapiens helicase (DNA) B (HELB), mRNA |
| 444 | 00800_597_G24 | | |
| 445 | 00800_597_I14 | NM_016592 | Homo sapiens GNAS complex locus (GNAS), transcript variant 4, mRNA |
| 446 | 00800_597_K23 | XM_001128735 | PREDICTED: Homo sapiens zinc finger protein 154 (pHZ-92) (ZNF154), mRNA |
| 447 | 00800_597_N16 | NT_011515 | Homo sapiens chromosome 21 genomic contig, reference assembly |
| 448 | 00800_599_C17 | NM_005654 | Homo sapiens nuclear receptor subfamily 2, group F, member 1 (NR2F1), mRNA |
| 449 | 00800_599_I11 | NM_003827 | Homo sapiens N-ethylmaleimide-sensitive factor attachment protein, alpha |
| | | | (NAPA), mRNA |
| 450 | 00800_599_N21 | NM_001069 | Homo sapiens tubulin, beta 2A (TUBB2A), mRNA |
| 451 | 00800_599_P10 | NM_015125 | Homo sapiens capicua homolog (Drosophila) (CIC), mRNA |
| 452 | 00800_600_C17 | NM_030567 | Homo sapiens proline rich 7 (synaptic) (PRR7), mRNA |
| 453 | 00800_600_C22 | NT_010542 | Homo sapiens chromosome 16 genomic contig, reference assembly |
| 454 | 00800_600_D09 | NM_018200 | Homo sapiens high-mobility group 20A (HMG20A), mRNA |
| 455 | 00800_600_I08 | | |
| 456 | 00800_600_K05 | NT_026446 | Homo sapiens chromosome 15 genomic contig, reference assembly |
| 457 | 00800_601_G06 | NM_001002246 | Homo sapiens APC11 anaphase promoting complex subunit 11 homolog (yeast) |
| | | | (ANAPC11), transcript variant 4, mRNA |
| 458 | 00800_602_A21 | NM_001002029 | Homo sapiens complement component 4B (Childo blood group) (C4B), mRNA |
| 459 | 00800_602_E13 | | |
| 460 | 00800_602_N08 | NT_028392 | Homo sapiens chromosome 20 genomic contig, reference assembly |
| 461 | 00800_603_N12 | NW_923907 | Homo sapiens chromosome 8 genomic contig, alternate assembly |
| | | | (based on Celera assembly) |
| 462 | 00800_603_O13 | | |
| 463 | 00800_533_J02 | NM_000992 | Homo sapiens ribosomal protein L29 (RPL29), mRNA |
| 464 | 09016_001_O18 | NM_003655 | Homo sapiens chromobox homolog 4 (Pc class homolog, Drosophila) |
| | | | (CBX4), mRNA |
| 465 | 09016_003_D06 | NM_024309 | Homo sapiens TNFAIP3 interacting protein 2 (TNIP2), mRNA |
| 466 | 09016_005_D22 | NM_004418 | Homo sapiens dual specificity phosphatase 2 (DUSP2), mRNA |
| 467 | 09016_005_G11 | NT_004836 | Homo sapiens chromosome 1 genomic contig, reference assembly |
| 468 | 09016_005_P19 | NT_021937 | Homo sapiens chromosome 1 genomic contig, reference assembly |
| 469 | 09016_007_F19 | NM_006753 | Homo sapiens surfeit 6 (SURF6), mRNA |
| 470 | 09016_007_P19 | NM_000972 | Homo sapiens ribosomal protein L7a (RPL7A), mRNA |
| 471 | 09016_008_D06 | NT_011255 | Homo sapiens chromosome 19 genomic contig, reference assembly |
| 472 | 09016_011_K20 | NM_004418 | Homo sapiens dual specificity phosphatase 2 (DUSP2), mRNA |
| 473 | 09016_014_N10 | NM_002405 | Homo sapiens manic fringe homolog (Drosophila) (MFNG), mRNA |
| 474 | 09016_016_I21 | NT_033903 | Homo sapiens chromosome 11 genomic contig, reference assembly |
| 475 | 09016_019_M11 | NM_032251 | Homo sapiens coiled-coil domain containing 88 (CCDC88), mRNA |
| 476 | 09016_021_K09 | NW_925517 | Homo sapiens chromosome 13 genomic contig, alternate assembly |
| | | | (based on Celera assembly) |
| 477 | 09016_022_F21 | NM_001010850 | Homo sapiens fusion (involved in t(12; 16) in malignant liposarcoma) |
| | | | (FUS), transcript variant 2, mRNA |
| 478 | 09016_022_O01 | NM_023008 | Homo sapiens hypothetical protein FLJ12949 (FLJ12949), transcript |
| | | | variant 1, mRNA |
| 479 | 09016_025_K08 | NT_026437 | Homo sapiens chromosome 14 genomic contig, reference assembly |
| 480 | 09016_028_N19 | NM_004418 | Homo sapiens dual specificity phosphatase 2 (DUSP2), mRNA |
| 481 | 09016_031_H22 | NT_011255 | Homo sapiens chromosome 19 genomic contig, reference assembly |
| 482 | 09016_031_J04 | NM_002360 | Homo sapiens v-maf musculoaponeurotic fibrosarcoma oncogene homolog K (avian) |
| | | | (MAFK), mRNA |
| 483 | 09016_031_J22 | NM_004593 | Homo sapiens splicing factor, arginine/serine-rich 10 (transformer 2 homolog, |
| | | | Drosophila) (SFRS10), mRNA |
| 484 | 09016_033_P14 | NM_018690 | Homo sapiens apolipoprotein B48 receptor (APOB48R), mRNA |
| 485 | 09016_038_K20 | NT_022184 | Homo sapiens chromosome 2 genomic contig, reference assembly |
| 486 | 09016_038_O04 | NM_002154 | Homo sapiens heat shock 70 kDa protein 4 (HSPA4), transcript variant 1, mRNA |
| 487 | 09017_001_D24 | NM_000701 | Homo sapiens ATPase, Na+/K+ transporting, alpha 1 polypeptide (ATP1A1), |
| | | | transcript variant 1, mRNA |
| 488 | 09017_002_B14 | NM_030665 | Homo sapiens retinoic acid induced 1 (RAI1), mRNA |
| 489 | 09017_003_C06 | NW_924951 | Homo sapiens chromosome 11 genomic contig, alternate assembly |
| | | | (based on Celera assembly) |
| 490 | 09017_003_I21 | NT_004487 | Homo sapiens chromosome 1 genomic contig, reference assembly |
| 491 | 09017_004_I18 | | |
| 492 | 09017_004_I24 | NT_026970 | Homo sapiens chromosome 2 genomic contig, reference assembly |
| 493 | 09017_005_A07 | NT_022184 | Homo sapiens chromosome 2 genomic contig, reference assembly |
| 494 | 09017_006_B18 | NM_001035518 | Homo sapiens centrosomal protein 250 kDa (CEP250), transcript variant 2, mRNA |
| 495 | 09017_007_J20 | NT_010783 | Homo sapiens chromosome 17 genomic contig, reference assembly |
| 496 | 09017_007_K23 | NT_011255 | Homo sapiens chromosome 19 genomic contig, reference assembly |
| 497 | 09017_008_K16 | NM_020524 | Homo sapiens pre-B-cell leukemia transcription factor interacting protein 1 |
| | | | (PBXIP1), mRNA |
| 498 | 09017_012_F22 | NM_023008 | Homo sapiens hypothetical protein FLJ12949 (FLJ12949), transcript variant 1, |
| | | | mRNA |
| 499 | 09017_013_D24 | NM_020524 | Homo sapiens pre-B-cell leukemia transcription factor Interacting protein 1 |
| | | | (P8XIP1), mRNA |
| 500 | 09017_013_E08 | NM_020524 | Homo sapiens pre-B-cell leukemia transcription factor interacting protein 1 |
| | | | (PBX1P1), mRNA |
| 501 | 09017_013_N01 | NT_028437 | Homo sapiens chromosome 14 genomic contig, reference assembly |
| 502 | 09017_014_N01 | NM_004418 | Homo sapiens dual specificity phosphatase 2 (DUSP2), mRNA |
| 503 | 09017_015_F06 | NM_023008 | Homo sapiens hypothetical protein FLJ12949 (FLJ12949), transcript variant 1, |
| | | | mRNA |
| 504 | 09017_016_C21 | NM_002952 | Homo sapiens ribosomal protein S2 (RPS2), mRNA |
| 505 | 09017_016_J19 | NM_012138 | Homo sapiens apoptosis antagonizing transcription factor (AATF), mRNA |
| 506 | 09017_017_M18 | NM_020524 | Homo sapiens pre-B-cell leukemia transcription factor interacting protein 1 |
| | | | (PBXIP1), mRNA |
| 507 | 09017_019_N07 | NT_011520 | Homo sapiens chromosome 22 genomic contig, reference assembly |
| 508 | 09017_019_N10 | NM_023008 | Homo sapiens hypothetical protein FLJ12949 (FLJ12949), transcript variant 1, |
| | | | mRNA |
| 509 | 09017_020_O16 | NM_005572 | Homo sapiens lamin A/C (LMNA), transcript variant 2, mRNA |
| 510 | 09017_021_G14 | NT_010783 | Homo sapiens chromosome 17 genomic contig, reference assembly |
| 511 | 09017_021_H12 | NM_173551 | Homo sapiens ankyrin repeat and sterile alpha motif domain containing 6 (ANKS6), |
| | | | mRNA |
| 512 | 09017_023_A19 | NT_011255 | Homo sapiens chromosome 19 genomic contig, reference assembly |
| 513 | 09017_023_L01 | NT_004559 | Homo sapiens chromosome 1 genomic contig, reference assembly |
| 514 | 09017_023_N16 | NM_001400 | Homo sapiens endothelial differentiation, sphingolipid G-protein-coupled |
| | | | receptor, 1 (EDG1), mRNA |
| 515 | 09017_024_D07 | NT_022517 | Homo sapiens chromosome 3 genomic contig, reference assembly |
| 516 | 09017_024_G15 | NT_021937 | Homo sapiens chromosome 1 genomic contig, reference assembly |
| 517 | 09017_025_P19 | NM_004418 | Homo sapiens dual specificity phosphatase 2 (DUSP2), mRNA |
| 518 | 09017_026_C11 | NM_012138 | Homo sapiens apoptosis antagonizing transcription factor (AATF), mRNA |
| 519 | 09017_026_C13 | NT_011109 | Homo sapiens chromosome 19 genomic contig, reference assembly |
| 520 | 09017_026_J08 | NT_022184 | Homo sapiens chromosome 2 genomic contig, reference assembly |
| 521 | 09017_028_O09 | NT_033903 | Homo sapiens chromosome 11 genomic contig, reference assembly |
| | | | coiled-coil domain containing 88 |
| 522 | 09017_029_I07 | XM_001134280 | PREDICTED: Homo sapiens v-maf musculoaponeurotic fibrosarcoma oncogene homolog |
| | | | (avian), transcript variant 2 (MAF), mRNA |
| 523 | 09017_033_C14 | NM_001034025 | Homo sapiens endoplasmic reticulum protein 29 (ERP29), transcript variant 2, |
| | | | mRNA |
| 524 | 09017_035_P22 | NM_020524 | Homo sapiens pre-B-cell leukemia transcription factor interacting protein 1 |
| | | | (PBXIP1), mRNA |
| 525 | 09017_037_B10 | NM_016111 | Homo sapiens TEL2, telomere maintenance 2, homolog (S. cerevisiae) |
| | | | (TELO2), mRNA |
| 526 | 09017_039_E14 | NM_138639 | Homo sapiens BCL2-like 12 (proline rich) (BCL2L12), transcript variant 1, mRNA |
| 527 | 09017_009_B10 | NM_001025100 | Homo sapiens myelin basic protein (MBP), transcript variant 8, mRNA |

1. A method for the diagnosis of multiple sclerosis, comprising determining at least one marker sequence of a cDNA comprising a sequence selected from the group SEQ 1-395 or respectively a protein coding therefor or respectively a partial sequence or fragment thereof from a patient to be examined.

2. The method of claim 1, wherein at least 2 to 5 or 10, preferably 30 to 50 marker sequences or 50 to 100 or more marker sequences from a patient to be examined are determined.

3. The method of claim 1, wherein SEQ 1-20 or SEQ 21-50 or SEQ 51-100 or respectively a protein coding therefor or respectively a pmiial sequence or a fragment thereof is determined from a patient to be examined are determined.

4. The method of claim 1, wherein the determination is carried out by means of in vitro diagnosis.

5. The method of claim 1, wherein the marker sequences are applied onto a solid support, in particular a filter, a membrane, a magnetic or fluorophore-labeled bead, a silica wafer, glass, metal, ceramics, plastics, a chip, a target for mass spectrometry or a matrix.

6. The method of claim 1, comprising
a. contacting a solid support having applied thereon at least one marker sequence of a cDNA selected from the group SEQ 1-395 or respectively a protein coding therefor or respectively a partial sequence or fragment thereof, with body fluid or tissue extract of a patient; and
b. detecting an interaction of the body fluid or tissue extract with the marker sequences on the solid support.

7. The method of claim 1, wherein the cDNA comprises the sequence set forth in SEQ ID NO: 78.

8. A method for the stratification, in particular risk stratification or therapy control of a patient with multiple sclerosis, comprising determining at least one marker sequence of a cDNA selected from the group SEQ 1-395 or respectively a protein coding therefor or respectively a partial sequence or fragment thereof from a patient to be examined.

9. The method according to claim 8, wherein the stratification or the therapy control covers decisions for the treatment and therapy of the patient, in particular the hospitalization of the patient, the use, effect and/or dosage of one or more drugs, a therapeutic measure or the monitoring of a course of the disease and the course of therapy, etiology or classification of a disease together with prognosis.

10. The method according to claim 8, wherein the cDNA comprises the sequence set forth in SEQ ID NO: 78.

11. An arrangement of marker sequences containing at least one marker sequence of a cDNA selected from the group SEQ 1-395 or respectively a protein coding therefor.

12. The arrangement according to claim 11, characterized in that at least 2 to 5 or 10, preferably 30 to 50 marker sequences or 50 to 100 or more marker sequences are contained.

13. The arrangement according to claim 11, characterized in that the marker sequences are present as clones.

14. The arrangement according to claim 11, characterized in that the marker sequences are applied to a solid support.

15. The arrangement according to claim 11, wherein the cDNA comprises the sequence set forth in SEQ ID NO: 78.
